(19) United States
(12) Patent Application Publication
(10) Pub. No.: US 2014/0086389 A1
(43) Pub. Date: Mar. 27, 2014

(54) METHOD AND APPARATUS FOR MORE ACCURATE POSITIONING OF DENTAL IMAGING EQUIPMENT

(71) Applicants: Seung H. BAEK, La Habra, CA (US); Gun Pyo PARK, Fullerton, CA (US)

(72) Inventors: Seung H. BAEK, La Habra, CA (US); Gun Pyo PARK, Fullerton, CA (US)

(21) Appl. No.: 14/025,715

(22) Filed: Sep. 12, 2013

Related U.S. Application Data
(60) Provisional application No. 61/699,870, filed on Sep. 12, 2012.
(60) Provisional application No. 61/699,872, filed on Sep. 12, 2012.
(60) Provisional application No. 61/710,919, filed on Oct. 8, 2012.

(51) Int. Cl. A61B 6/00; A61B 6/14

(52) U.S. Cl. CPC A61B 6/587; A61B 6/145
USPC 378/62; 378/147

(57) ABSTRACT

An apparatus for positioning an X-ray imaging system. The apparatus includes an intraoral sensor sized and configured for placement within a human mouth, the intraoral sensor having one or more sensing elements for detecting X-rays, the intraoral sensor further having a signal generation element. Also included is a collimator unit having an X-ray collimator and first and second signal detection elements spaced apart from each other with the X-ray collimator positioned therebetween, the first and second signal detection elements spaced apart from each other, to thereby facilitate aligning of the collimator unit with the one or more sensing elements according to detection of signals from the signal generation element. Further included is an indicator unit located on the collimator unit, the indicator unit configured to display a determination as to whether the collimator unit is properly aligned with respect to the intraoral sensor.

### CROSS REFERENCE TO RELATED APPLICATIONS

This application claims priority to, and the benefit of, U.S. Provisional Application No. 61/699,870 filed on Sep. 12, 2012, U.S. Provisional Application No. 61/699,872 filed on Sep. 12, 2012, and U.S. Provisional Application No. 61/710,919 filed on Oct. 8, 2012, the contents of each of which are hereby incorporated by reference in their entireties.

### BRIEF DESCRIPTION

Embodiments of the invention relate generally to dental imaging equipment. More specifically, embodiments of the invention relate to systems and methods for more accurate positioning of dental imaging equipment.

### BACKGROUND

The currently accepted method of taking X-ray images of teeth has remained largely unchanged for many years. In essence, an X-ray machine employs a movable collimator and an X-ray detector. The detector is typically placed in the patient's mouth against his or her teeth, and a technician moves the collimator close to the patient's mouth, so that X-rays from the collimator pass through the patient's teeth and other oral tissues, striking the detector and thereby generating dental images.

This approach has certain challenges, however. The collimator must be aimed in the correct direction and be positioned at the correct distance or the X-rays will partly or entirely miss the detector, or be unfocused, creating poor images. For example, inaccurate aim and distancing of the collimator may produce cone-cut images, while excessive distance can also result in reduced image quality. Such poor images may require the technician to take additional pictures, thereby exposing the patient to more X-ray radiation.

To ameliorate this situation, positioning guides are currently employed to help in proper aiming of the collimator. FIGS. 1 and 2 illustrate two such conventional positioners. FIG. 1 illustrates a guide in which film is held at the vertical plate shown on the left-hand side of the Figure. The film and guide are placed in the patient's mouth, and the remainder of the guide (that shown at the right-hand side of FIG. 1) extends out from the patient's mouth, helping the technician to estimate the location of the film. FIG. 2 shows another conventional guide, this one having a vertical dark-colored plate upon which the X-ray detector is placed, and a ring-shaped structure centered at or near the midpoint of the location at which the detector is to be placed. The plate and detector are placed in the patient's mouth and held by his or her clenched teeth, while the technician aims the collimator so that the X-ray beam is directed through the ring. Such conventional positioning guides are, however, somewhat bulky and cumbersome to use, as well as being difficult and/or uncomfortable for patients to hold in their mouths.

Other conventional positioning systems are illustrated in, for example, U.S. Pat. Nos. 4,223,228 and 5,463,669 (“the '228 and '669 patents”), which are hereby incorporated by reference. Such systems employ a single magnet placed behind the X-ray film, and magnetic field detectors within the collimator tube. Among other drawbacks however, such systems also employ large numbers of sensors (eight, in the case of both the '228 and '669 patents), adding to complexity and expense. The systems of the '228 and '669 patents further employ separate display or readout units located remote from the collimator, which requires users to take their eyes away from the collimator unit during aligning, thus reducing alignment accuracy. The use of a separate display unit also adds to the complexity and bulkiness of the device. Users must also take their eyes off the collimator unit to look at the display, making the alignment process more difficult and less accurate.

### SUMMARY

The invention can be implemented in many ways. In one embodiment, an X-ray system comprises an intraoral sensor, a collimator unit, and an indicator unit. The intraoral sensor is sized and configured for placement within a human mouth, and has one or more sensing elements for detecting X-rays, as well as a signal generation element. The collimator unit has an X-ray collimator and first and second signal detection elements spaced apart from each other with the X-ray collimator positioned therebetween, the first and second signal detection elements spaced apart from each other to correspond to the signal generation element, to thereby facilitate aligning of the collimator unit with the one or more sensing elements according to detection of signals from the signal generation element. The indicator unit is located on the collimator unit, and is configured to display a determination as to whether the collimator unit is properly aligned with respect to the intraoral sensor.

The signal generation element can be a magnet, and the signals from the signal generation element can comprise magnetic fields. The magnet can also be one of a rare earth magnet and an electromagnet.

The first and second signal detection elements can each be configured to detect a magnitude and/or a direction of the magnetic field of the signal generation element.

The first and second signal detection elements can be spaced apart from each other by a distance determined at least partially according to a predetermined distance between the collimator unit and the signal generation element.

Each of the signal detection elements can be oriented toward the signal generation element.

The indicator unit can be further configured to display one or more instructions for more properly aligning the collimator unit with respect to the intraoral sensor.

The indicator unit can comprise one or more of a visual display and a speaker.

The intraoral sensor can be a sensor holder to which the signal generation element can be attached. The intraoral sensor may be further configured so that the one or more sensing elements can be removably affixed thereto.

In another embodiment, a dental imaging system can comprise an intraoral sensor sized and configured for placement within a human mouth, the intraoral sensor having one or more sensing elements for detecting imaging signals, the intraoral sensor further having a signal generation element positioned therein, the signal generation element configured to emit positioning signals. The dental imaging system can also include an emission unit having an emitter configured to emit the imaging signals, the emission unit further having at least two signal detection elements each positioned proximate to the emitter and positioned to correspond to the signal generation element so as to thereby facilitate aligning of the emission unit according to detection of the positioning signals. Also included may be an indicator unit located on the emission unit, the indicator unit configured to display an indication as to whether the emission unit is properly aligned with respect to the intraoral sensor.

The two or more signal detection elements can be spaced apart from each other with the emitter positioned therebetween.

### BRIEF DESCRIPTION OF THE DRAWINGS

For a better understanding of the invention, reference should be made to the following detailed description taken in conjunction with the accompanying drawings, in which:

FIG. 1 illustrates an example of a conventional X-ray collimator positioning guide;

FIG. 2 illustrates another example of a conventional X-ray collimator positioning guide;

FIG. 3 is a cross-sectional view of a patient's mouth illustrating operation of an X-ray positioning system of embodiments of the present invention;

FIG. 4 is a magnified view of another embodiment of an X-ray positioning system; and

FIGS. 5A-5C are isometric, cross-sectional, and cutaway views, respectively, illustrating further details of an intraoral sensor of embodiments of the present invention.

FIG. 6 is a picture of a modified sensor holder for use in conjunction with embodiments of the present invention.

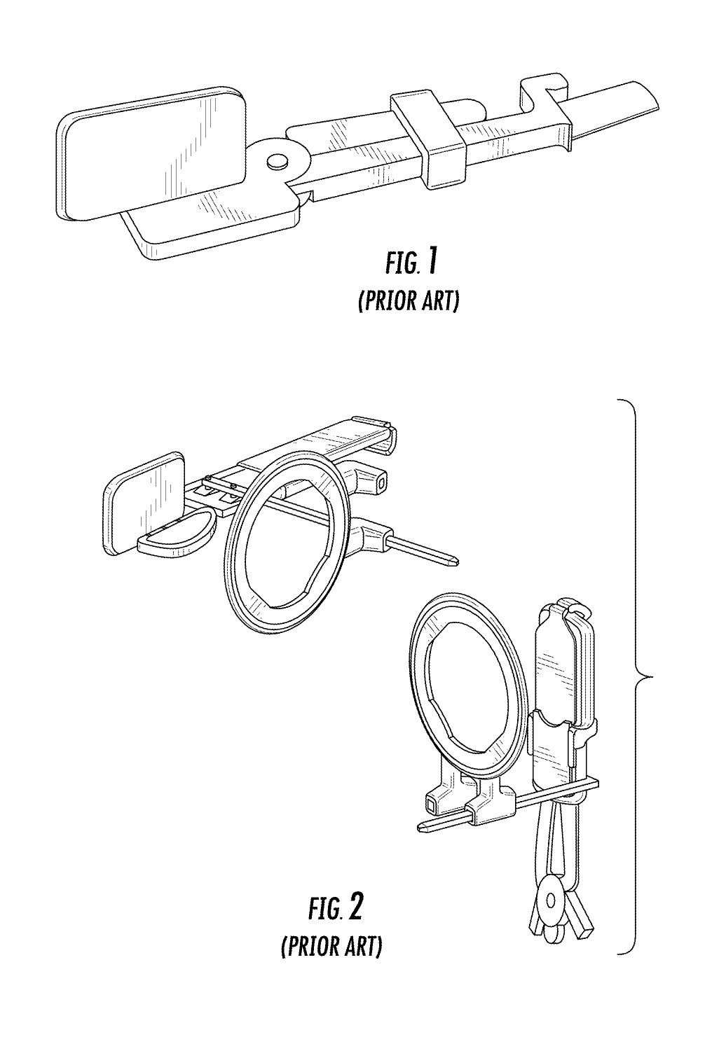

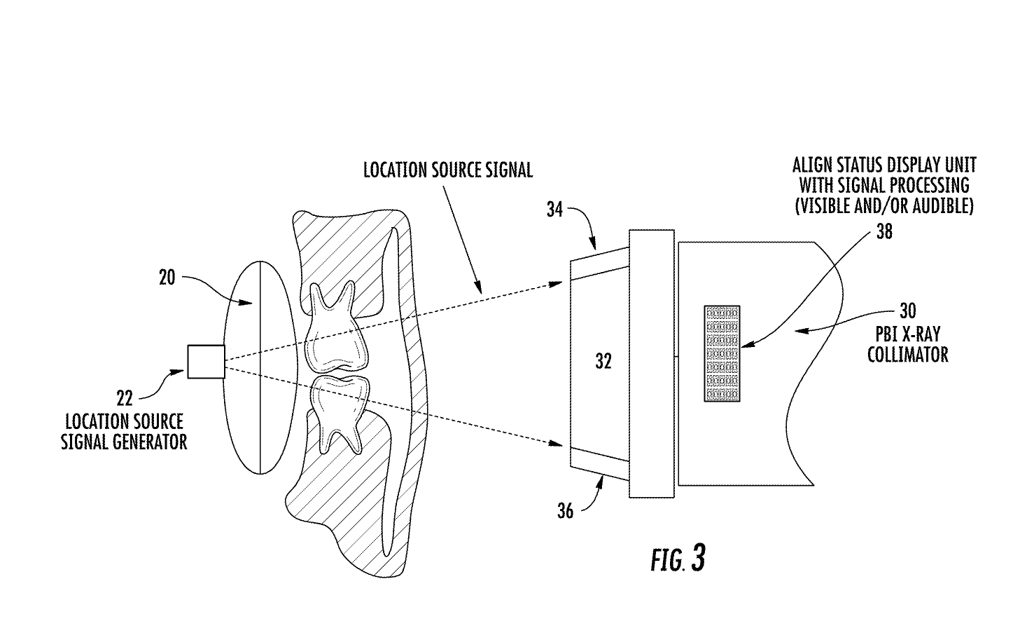

Like reference numerals refer to corresponding parts throughout the drawings. The various Figures are not necessarily to scale.

### DETAILED DESCRIPTION

In one embodiment, the invention is a dental imaging system designed to be more easily and correctly positioned, so as to yield more reliably accurate dental images. The system includes an intraoral sensor to be placed in the patient's mouth, where a magnetic field generator is placed within or on the sensor. A collimator unit has detectors for detecting this magnetic field, where the detectors are spaced apart from each other at a distance corresponding to the magnetic field generators of the sensor. In this manner, a user can readily manipulate the collimator until each detector detects magnetic field signals from the field generator, thus properly aligning the collimator with the sensor. Furthermore, a display indicating alignment status is located on the collimator unit itself, so that users need not take their eyes off the collimator unit during alignment. Both of these aspects allow for quicker and more accurate alignment, thus more reliably yielding accurate X-ray images. Different configurations and numbers of both the magnetic field generators and the detectors are also contemplated.

FIG. 3 is a cross-sectional view of a patient's mouth illustrating operation of an X-ray positioning system of embodiments of the present invention. Here, an imaging system 10 includes an intraoral sensor 20 and collimator housing 30. The intraoral sensor 20 is configured and sized for placement in a human mouth as shown, and contains an X-ray detector or X-ray film (not shown), as well as a magnetic field generator 22. The X-ray detector or film is placed within the intraoral sensor 20 in a central portion of the device, and the magnetic field generator 22 may be placed anywhere within the sensor 20 or on the surface thereof, as shown. The magnetic field generator 22 can be, for example, a magnet such as a simple magnet (i.e. pieces of magnetic material) or rare earth magnet, electromagnet, or any other object that generates, or is capable of generating, a magnetic field.

The collimator housing 30 contains an X-ray collimator (not shown) for directing X-rays, a tube 32 through which X-rays from the collimator are emitted, two detectors 34, 36 that detect magnetic fields, and a processor and display unit 38. The detectors 34 are located on opposite sides of the tube 32 and are spaced apart and oriented to correspond to the magnetic field generator 22 of the intraoral sensor 20, as shown. The detectors 34 can be any magnetic field detectors, such as Hall Effect sensors capable of detecting the strength and direction of a magnetic field.

The processor and display unit 38 is connected to the detectors 34, 36 and contains a processor as well as an electronic display such as light emitting diodes, a display screen, or the like. The display lights up or emits some other indicator (e.g., an audible sound from a speaker) when the collimator housing 30 is properly aligned with the intraoral sensor 20. More specifically, the unit 38 contains a processor that receives data output from the detectors 34, 36, and that is programmed to trigger the display when the detectors 34, 36 both indicate that they are aligned with (e.g., both detect magnetic fields emanating from) the magnetic field generator 22. Indication of alignment can be by any criteria. For example, the sensor 20 and housing 30 can be deemed to be aligned when each detector 34, 36 detects a magnetic field intensity above some predetermined amount. Alternatively, alignment may be determined according to both magnetic field strength and direction measurements from each detector 34, 36. That is, proper alignment can be deemed to have occurred when each detector 34, 36 detects a sufficiently strong magnetic field and/or also detects a field vector oriented in a predetermined direction, e.g. oriented at a predetermined angle with respect to the length direction of that detector 34, 36. In this manner, alignment occurs when each detector 34, 36 is both sufficiently close to its corresponding magnet and is oriented at a predetermined angle thereto.

Notably, the processor and display unit 38 is located on the collimator housing 30. Thus, a visual and/or audible indication of alignment can be displayed on the housing 30 itself, so that users need not take their eyes off the collimator housing 30 and/or patient during alignment. By offering real-time feedback within the field of vision of an operator looking at the housing 30 or patient, quicker and more accurate alignment of the housing 30 to the sensor 20 can be achieved.

The processor and display unit 38 can display any indication of alignment that is desired. For example, the processor and display unit 38 can give audio/visual indicators of any or all of distance, position, and angular orientation, based on detector feedback. More specifically, determinations of magnetic field strength from each detector 34, 36 can indicate whether the housing 30 is located too far or too close to the sensor 20, while differential magnitudes and directions can indicate that the housing 30 must be translated in one or more directions, or rotated about some axis. For instance, low field strength readings from both detectors 34, 36 may indicate that the housing 30 is too far from the sensor 20, and must be moved closer. Similarly, differing magnitudes and directions may indicate misalignment (i.e., housing 30 pointed in the wrong direction), while correct directions but low magnitudes may indicate the need for the housing 30 to be translated, or moved laterally, in one or more directions. One of ordinary skill in the art will observe that determination of the degree of misalignment and mis-position (e.g., incorrect orientation and position) may be determined from multiple spaced-apart detectors 34, 36. The display unit 38 may be programmed to display corresponding instructions for proper alignment, based on the calculated degrees of misalignment and/or mis-positioning. For example, in real-time, the display unit 38 may display instructions to move the housing 30 closer to or farther from the patient's mouth, to turn the housing 30 in a particular direction or directions, to move the housing 30 up/down, etc. The display unit 38 may also emit a visual and/or audible indicator when sufficiently proper alignment has occurred. Alternatively, instructions displayed by the display unit 38 may simply be a visual and/or audible indicator of misalignment, without indicating what to do for more proper alignment. Embodiments of the invention contemplate the display of any such instructions, in any suitable manner.

In operation, the intraoral sensor 20 is first placed in a patient's mouth and positioned so that its X-ray film or detector is placed proximate to the portion of the mouth that is to be photographed. In FIG. 3, the sensor 20 is shown positioned so that an image of the teeth shown is to be taken. The collimator housing 30 is moved close to the patient's mouth and oriented so that tube 32 faces the intraoral sensor 20. The detectors 34, 36 determine the position and/or orientation of the housing 30 relative to the sensor 20, and the display unit 38 indicates whether the housing 30 is aligned properly or not, as well as optionally displaying instructions for adjusting the housing. The position of collimator housing 30 is then adjusted until detectors 34, 36 each detect a sufficiently high magnetic field strength, and/or detect a magnetic field having a particular direction. As can be seen in FIG. 3, because the detectors 34, 36 are spaced apart, the collimator housing 30 and sensor 20 are aligned with each other when both detectors 34, 36 simultaneously detect sufficiently strong magnetic fields (or, perhaps, sufficiently strong fields as well as field direction vectors that are mirror images of each other relative to the major axis of the collimator housing 30 and tube 32). Any field strength and/or detection criteria are contemplated.

When both detectors 34, 36 simultaneously detect a sufficiently strong magnetic field, and/or detect a magnetic field having a particular direction, the processor and display unit 38 determines that the sensor 20 and housing 30 are aligned, and emits an indication that alignment has occurred. As above, the indication can be an audible signal from a speaker, a visual signal displayed on a visual display, some combination of the two, or some other indicator of alignment.

In the embodiment of FIG. 3, two detectors 34, 36 are spaced apart from each other at either side of collimator tube 32, where they can determine alignment according to the detected strength and direction of the field emitted by field generator 22. Thus, as can be seen, simultaneous detection of sufficiently strong magnetic fields by both detectors 34, 36 indicates proper alignment.

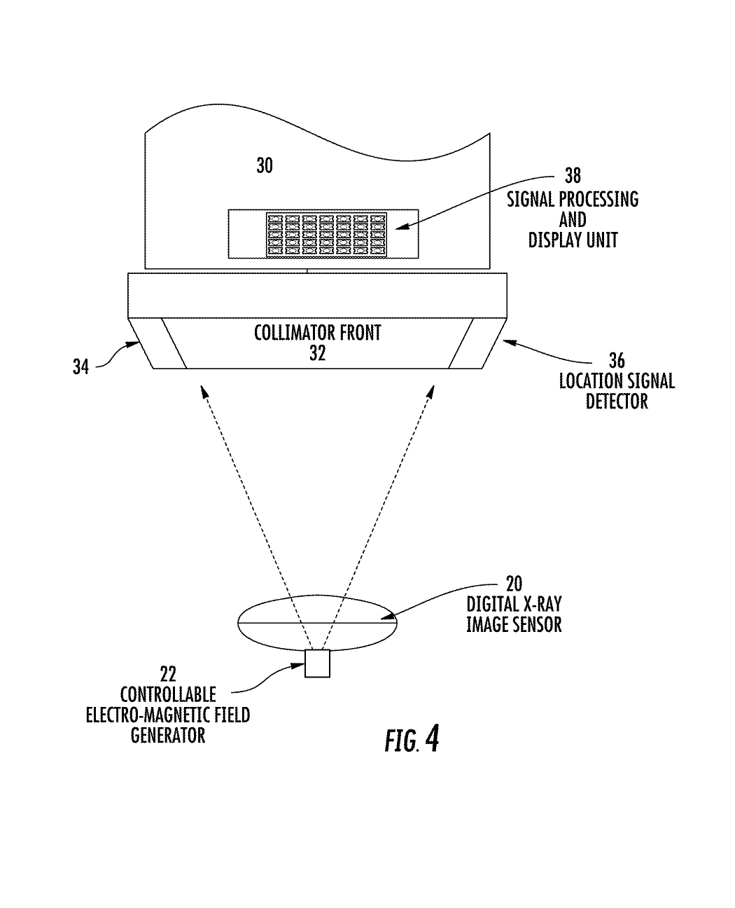

However, embodiments of the invention are not limited to the configuration in FIG. 3, and any spacings and orientations of the detectors 34, 36 and magnetic field generator 22 are contemplated, so long as they allow for proper alignment of the sensor 20 and collimator housing 30 according to readings from the detectors 34, 36. For example, as shown in FIG. 4, the detectors 34, 36 are spaced apart from each other, and each is oriented toward the detector 20. The angle of orientation can be determined by the distance between magnetic field generator 22 and detectors 34, 36 that is preferred for X-ray images of good quality. That is, the orientation is at least partly determined according to the distance between collimator housing 30 and sensor 20 that results in the best X-ray images. The detectors 34, 36 can thus be spaced any desired distance apart from each other, according to a desired predetermined distance between housing 30 and field generator 22. A larger distance may, for example, help improve accuracy. For instance, as the distance between detectors 34 and 36 increases, differences in field direction are more likely to be greater, and thus easier to detect. Greater distances between detectors 34, and 36 thus make it easier to determine alignment correctly, so long as the distance between detectors 34, 36 is not so great that the field strength is excessively low and thus hard to detect even when the collimator unit 30 is positioned correctly. Orienting the magnetic field generators 22, 24 at angles, and thus setting the detectors 34, 36 a predetermined distance apart from each other, may therefore be desirable in that it may allow for increased accuracy of the imaging system 10.

When field generator 22 is a magnet, it may be configured as a bar magnet with a major axis, and may be oriented parallel with, or perpendicular to, the direction of emission of X-rays. The detectors 34, 36 may also be oriented at a specified angle with respect to (in this case, pointing at) the magnet, as shown. In this configuration, the detected magnetic field would have a known direction with respect to each of the detectors 34, 36 when the sensor 20 and collimator housing 30 are properly oriented (i.e. are collinear). Similarly, the detected magnetic field would have a known intensity at each detector 34, 36 at proper alignment. Alignment can thus be verified when the detected field magnitude and/or direction match these values. Any appropriate direction is contemplated.

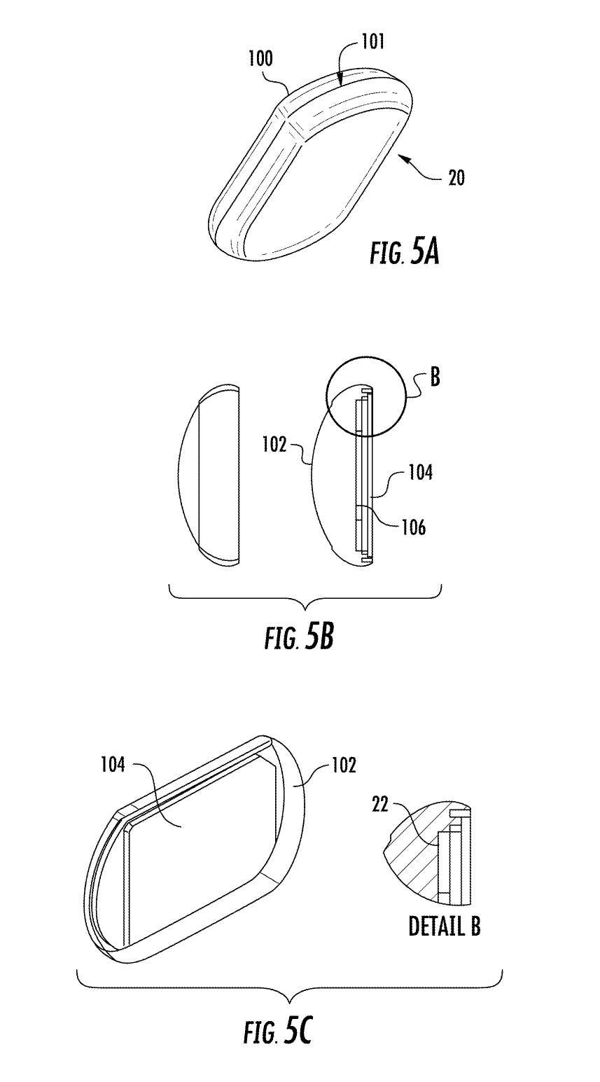

FIGS. 5A-5C are isometric, cross-sectional, and cutaway views, respectively, illustrating further details of intraoral sensor 20. The intraoral sensor 20 is designed to withstand the environment of a human mouth yet have a soft exterior to prevent discomfort, with waterproof polymer halves 100 and 102 coupled together along a seam 101. The two polymer halves 100 and 102 may be configured to be removably coupled together such as by press-fit or snap-fit joints, or one or both may be configured to be removably coupled to a plate 104. The plate 104 may be sized and configured to hold an X-ray film or detector 106, and may also be optionally configured to hold a magnetic field generator 22. The plate 104 may be shaped so that X-ray film/detector 106 can be removably pressed thereon, while magnetic field generators 22, if located within sensor 20, may be affixed to the plate 104 by, for example, an adhesive. In this manner, the X-ray film/detector 106 can be pressed onto plate 104 and then one or more of the two halves 100, 102 can be snapped onto the other, whereupon the sensor 20 can be placed in the mouth. When alignment and exposure are complete, the process is reversed, with the two halves 100, 102 being separated and the X-ray film/detector 106 removed, if necessary, for photo development.

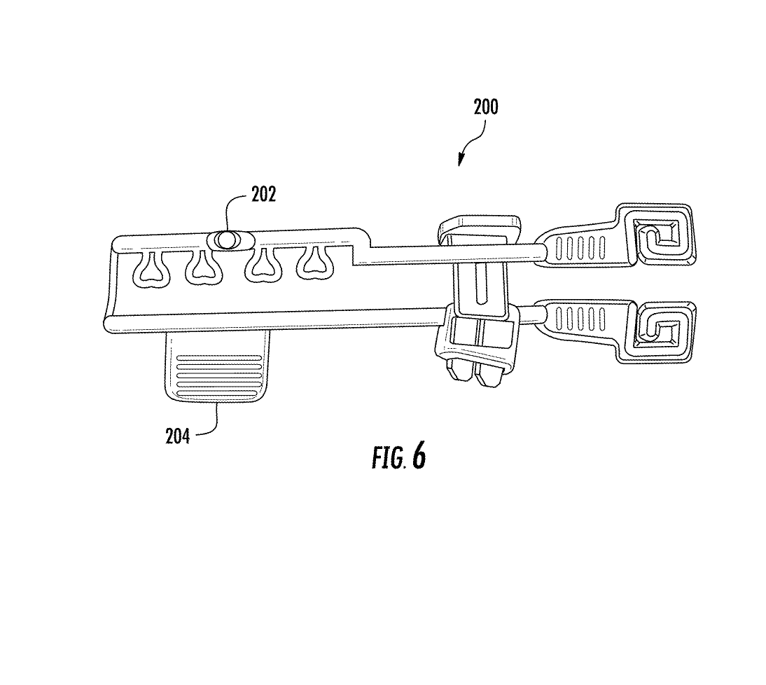

The intraoral sensor 20 can take on numerous forms consistent with embodiments of the invention. For instance, the intraoral sensor 20 can be similar to a conventional X-ray film or sensor holder but modified to hold one or more magnetic field generators. FIG. 6 illustrates a modified sensor holder for use in conjunction with embodiments of the present invention. Here, sensor holder 200 is similar to conventional sensor holders as can be observed, and has a bite wing 204 for the patient to bite down on, thus holding the sensor holder within the mouth. The sensor holder 200 is configured to removably hold X-ray film or some other X-ray sensor against the teeth when the bite wing 204 is bitten down on, as can be readily observed by one of ordinary skill in the art. The sensor holder 200 is also modified to carry a magnetic field generator, in this case a magnet 202. The sensor holder 200 is configured to hold a magnet 202 on its upper arm, as shown in FIG. 6. However, embodiments of the invention contemplate sensor holders 200 that can hold one or more magnets 202 at any suitable location within or on the sensor holder 200.

Any configuration and orientation of magnetic field generator 22 is contemplated, so long as it is capable of emitting magnetic fields of sufficient strength and in directions allowing for proper alignment according to detection by detectors 34, 36. Thus, the magnetic field generator 22 can be a flat structure, such as that shown in FIGS. 5B-5C (showing an alternative embodiment in which the field generator 22 is located at one end of the sensor 20 rather than at a central portion thereof), or structures of any other shape and/or orientation.

Furthermore, one of ordinary skill in the art will realize that the invention is not limited to the above described embodiments. For instance, while only one magnetic field generator 22 and only two detectors 34, 36 are shown, any number of generators and detectors is contemplated. For example, multiple magnetic field generators may be placed within sensor 20, and three detectors may be placed at corresponding positions around the collimator, so that alignment occurs when all three detectors detect magnetic fields from each of their corresponding field generators. The detectors and field generators may be placed radially equidistant from each other, with the X-ray film/detector in-between each of the field generators and the collimator or collimator tube in-between each of the detectors. Alternatively, the detectors and field generators may be placed non-equidistant from each other, as desired. Additionally, the generators may generate any type of signal besides that of a magnetic field. Any type of signal capable of safe emission through human tissue and reliable detection is contemplated. The detectors 34, 36 may also be placed anywhere on the housing 30 besides the tube 32, such as on some portion of the housing 30 that is outside the tube 32 and faces the sensor 20 when the housing 30 is properly aligned therewith.

The foregoing description, for purposes of explanation, used specific nomenclature to provide a thorough understanding of the invention. However, it will be apparent to one skilled in the art that the specific details are not required in order to practice the invention. Thus, the foregoing descriptions of specific embodiments of the present invention are presented for purposes of illustration and description. They are not intended to be exhaustive or to limit the invention to the precise forms disclosed. Many modifications and variations are possible in view of the above teachings. For example, any magnetic field generator may be employed, whether it be a magnet, electromagnet, or other; any other type of field or signal generator may also be employed. The detectors 34, 36 may also sense field intensity, direction, or some combination of both. The display unit 38 may determine alignment by any one or more of field intensity and direction measurements from any number of detectors located anywhere on housing 30, and may display any form of alignment indication to the user, as well as directions for more proper alignment if desired. Also, the various embodiments each have certain features that differ from those of other embodiments, and it is noted that the invention contemplates the mixing and matching of various features as desired. That is, further embodiments can be formed from the selection of various features from different embodiments. The embodiments were chosen and described in order to best explain the principles of the invention and its practical applications, to thereby enable others skilled in the art to best utilize the invention and various embodiments with

What is claimed is:

1. An X-ray system comprising:
an intraoral sensor sized and configured for placement within a human mouth, the intraoral sensor having one or more sensing elements for detecting X-rays, the intraoral sensor further having a signal generation element;
a collimator unit having an X-ray collimator and first and second signal detection elements spaced apart from each other with the X-ray collimator positioned therebetween, the first and second signal detection elements spaced apart from each other to correspond to the signal generation element, to thereby facilitate aligning of the collimator unit with the one or more sensing elements according to detection of signals from the signal generation element; and
an indicator unit located on the collimator unit, the indicator unit configured to display a determination as to whether the collimator unit is properly aligned with respect to the intraoral sensor.

2. The X-ray system of claim 1, wherein the signal generation element is a magnet, and the signals from the signal generation element comprise magnetic fields.

3. The X-ray system of claim 2, wherein the magnet is one of a rare earth magnet and an electromagnet.

4. The X-ray system of claim 2, wherein the first and second signal detection elements are each configured to detect a magnitude of the magnetic field of the signal generation element.

5. The X-ray system of claim 4, wherein the first and second signal detection elements are each configured to detect a direction of the magnetic field of the signal generation element.

6. The X-ray system of claim 1, wherein the first and second signal detection elements are spaced apart from each other by a distance determined at least partially according to a predetermined distance between the collimator unit and the signal generation element.

7. The X-ray system of claim 6, wherein each of the signal detection elements is oriented toward the signal generation element.

8. The X-ray system of claim 1, wherein the indicator unit is further configured to display one or more instructions for more properly aligning the collimator unit with respect to the intraoral sensor.

9. The X-ray system of claim 1, wherein the indicator unit comprises one or more of a visual display and a speaker.

10. The X-ray system of claim 1, wherein the intraoral sensor is a sensor holder to which the signal generation element can be attached, the intraoral sensor further configured so that the one or more sensing elements can be removably affixed thereto.

11. A dental imaging system comprising:
an intraoral sensor sized and configured for placement within a human mouth, the intraoral sensor having one or more sensing elements for detecting imaging signals, the intraoral sensor further having a signal generation element positioned therein, the signal generation element configured to emit positioning signals;
an emission unit having an emitter configured to emit the imaging signals, the emission unit further having at least two signal detection elements each positioned proximate to the emitter and positioned to correspond to the signal generation element so as to thereby facilitate aligning of the emission unit according to detection of the positioning signals; and
an indicator unit located on the emission unit, the indicator unit configured to display an indication as to whether the emission unit is properly aligned with respect to the intraoral sensor.

12. The dental imaging system of claim 11, wherein signal generation element is a magnet, and the positioning signals comprise magnetic fields.

13. The dental imaging system of claim 12, wherein the magnet is one of a rare earth magnet and an electromagnet.

14. The dental imaging system of claim 12, wherein each of the signal detection elements is configured to detect a magnitude of the magnetic field of the signal generation element.

15. The dental imaging system of claim 14, wherein each of the signal detection elements is configured to detect a direction of the magnetic field of the signal generation element.

16. The dental imaging system of claim 11, wherein at least two of the signal generation elements are spaced apart from each other by a distance determined at least partially according to a predetermined distance between the emission unit and the signal generation element.

17. The dental imaging system of claim 16, wherein each of the signal detection elements is oriented toward the signal generation element.

18. The dental imaging system of claim 11, wherein the two or more signal detection elements are spaced apart from each other with the emitter positioned therebetween.

19. The dental imaging system of claim 11, wherein the indicator unit is further configured to display one or more instructions for more properly aligning the emission unit with respect to the intraoral sensor.

20. The dental imaging system of claim 19, wherein the indicator unit comprises one or more of a visual display and a speaker.

21. The dental imaging system of claim 11, wherein the intraoral sensor is a sensor holder to which the signal generation element can be attached, the intraoral sensor further configured so that the one or more sensing elements can be removably affixed thereto.
